(19) United States
(12) Patent Application Publication
(10) Pub. No.: US 2025/0205129 A1
(43) Pub. Date: Jun. 26, 2025

(54) SYSTEM FOR HAIR CARE

(71) Applicant: Kenvue Brands LLC, Summit, NJ (US)

(72) Inventors: Ana Carolina Nogueira Braverman, Jersey City, NJ (US); Daniel Knatz, Sarasota, FL (US); Frank C. Sun, Belle Mead, NJ (US)

(21) Appl. No.: 18/985,273

(22) Filed: Dec. 18, 2024

Related U.S. Application Data
(60) Provisional application No. 63/612,648, filed on Dec. 20, 2023.

(51) Int. Cl. A61K 8/44; A61Q 5/02; A61Q 5/12

(52) U.S. Cl. CPC A61K 8/44; A61Q 5/02; A61Q 5/12; A61K 2800/52; A61K 2800/5426

(57) ABSTRACT

A system, or kit, for hair care comprising a cleansing formulation and a hair conditioning formulation. The system includes a hair cleansing formulation comprising a surfactant mixture comprising at least an amphoteric surfactant, an anionic surfactant, and a non-ionic surfactant; and water. Active materials present in the surfactant mixture amount for at least 10%, in weight %, of the hair cleansing formulation. The system further includes a hair conditioning formulation comprising a conditioning agent, an emollient, and a non-ionic surfactant.

### CROSS-REFERENCE TO RELATED APPLICATION

This application claims the benefit of U.S. Provisional Application No. 63/612,648 filed Dec. 20, 2023, the contents of which is incorporated herein by reference in its entirety.

### BACKGROUND

Hair cleansers and hair conditioners are often used in the same hair care routine therefore being part of a kit or system for hair care. Many consumers prefer products that meet several characteristics. For example, many consumers prefer a hair cleanser and hair conditioner with benefits that include a pleasant texture and viscosity within an appropriate range of thickness, strong lathering properties when applied and massaged on wet hair, and an appropriate mildness to prevent damages to the hair fiber, by stripping the proteins and natural oils present on the hair fibers, and for the scalp skin. The combination of hair cleanser and hair conditioner should prepare the hair for combing, reduce the hair breakage, reduce hair split ends, and provide volume and softness to the dried hairs.

In addition, consumers are increasingly concerned about the environmental sustainability of the products they purchase and would prefer hair cleanser and hair conditioner formulations with the highest possible proportion of renewable ingredients. Ideal hair cleansers and hair conditioners address all of these requirements, while also meeting manufacturing constraints of the formulation such as stability, pH, and viscosity.

### BRIEF DESCRIPTION OF THE DRAWINGS

The present description will be better understood from the following detailed description read in light of the accompanying drawings, wherein:

FIG. 1 illustrates values extracted from the ATR-FTIR spectra for the total lipid content of example formulations as described herein

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Various implementations of the present disclosure described herein are directed to systems and methods for hair care. In one implementation, a system is provided. The system includes a hair cleansing formulation and a hair conditioning formulation. The hair cleansing formulation comprises a surfactant mixture comprising at least: an amphoteric surfactant, an anionic surfactant and a non-ionic surfactant; and water. The active materials present in the surfactant mixture amount for at least 10%, in weight %, of the hair cleansing formulation. The hair conditioning formulation comprises at least one conditioning agent; at least one emollient; and at least one non-ionic surfactant.

In another implementation, a method is provided. The method includes treating human hair with the system for hair care by applying the hair cleansing formulation to the human hair, rinsing the hair cleansing formulation from the human hair; and applying the hair conditioning formulation to the human hair.

In another implementation, a method is provided. The method includes reducing split ends in human hair by contacting a subject hair with the system for hair care.

In another implementation, a method is provided. The method includes reducing human hair breakage by contacting a subject hair with the system for hair care.

### DETAILED DESCRIPTION

The various implementations and examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all examples.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54. All percentages (%) are by weight unless otherwise specified herein.

In some examples, the present disclosure provides a system for hair care comprising a hair cleansing formulation and a hair conditioning formulation. The hair cleansing formulation comprises a surfactant mixture comprising at least an amphoteric surfactant, an anionic surfactant, and a non ionic surfactant; and water. The active materials present in the surfactant mixture amount for at least 10%, in weight %, of the hair cleansing formulation. The hair conditioning formulation comprises: a conditioning agent preferably selected among alkyl quaternary ammonium; amides of fatty acid and amino alkyl; and silicones, or mixture thereof, an emollient preferably selected among esters of fatty acid such as Isopropyl myristate and Coco caprylate/caprate; and a non ionic surfactant, preferably selected among ethoxylated alcohols, sorbitan olivate, propylene glycol isostearate, glycol stearate, sorbitan sesquioleate, glyceryl stearate, lecithin, sorbitan oleate, sorbitan, monostearate NF, sorbitan stearate, sorbitan isostearate, steareth-2, Steareth-20, oleth-2, glyceryl laurate ceteth-2, PEG-30 dipolyhydroxystearate, glyceryl stearate SE, sorbitan stearate, sucrose cocoate, PEG-4 dilaurate, methyl glucose sesquistearate, PEG-8 dioleate, sorbitan laurate, PEG-40 sorbitan peroleate, laureth-4, glycerol monostearate, propylene glycol ester of myristic acid, ester of palmitic acid, monoglyceride ester of myristic acid, monoglyceride ester of palmitic acid, monoglyceride ester stearic acid, monoglyceride ester of oleic acid, ester of myristic acid, sorbitan ester of palmitic acid, sorbitan ester of stearic acid, sorbitan ester of oleic acid, ester of stearic acid, ester of oleaic acid, sorbitan sesquioleate, polyoxyethylenes sorbitol, oleate, sorbitan monooleate, propylene glycol monolaurate, propylene glycol monolaurate, propylene glycol fatty acid ester, sorbitan monostearate, sorbitan monostearate, diethylene glycol monostearate, diethylene glycol fatty acid ester, polyoxyethylene sorbitol beeswax derivative, diethylene glycol fatty acid ester, diethylene glycol monolaurate (soap free), sorbitan monopahnitate, sorbitan monopahnitate, polyoxyethylene dioleate, tetraethylene glycol monooleate, polyoxypropylene mannitol dioleate, polyoxyethylene sorbitol lanolin, oleate derivative, polyoxyethylene sorbitol lanolin derivative, polyoxypropylene stearate, and sorbitan monolaurate. In some examples at least one non-ionic surfactant is selected among alkoxylated alcohols, ethoxylated alcohols, or combinations thereof. In some examples, at least one non-ionic surfactant is selected among: Steareth-20, polysorbate-20, Ceteareth-20, Steareth-10 or other C16-C18 ethoxylated fatty alcohols with a number of ethoxylation from 10 to 30, or combinations thereof. In some examples, the at least one conditioning agent present in the hair conditioning formulation may be selected among Behentrimonium chloride, Stearamidopropyl Dimethylamine, dimethicone or mixture thereof.

The formulation described herein contain water as a cosmetically-acceptable carrier. As used herein, the term “cosmetically-acceptable carrier” means a carrier that is suitable for use in contact with the skin without undue toxicity, incompatibility, instability, irritation, allergic response, and the like. Water may be present in an amount ranging from about 50, 55, 60, 65, 70, 75, 80, 85, or 90% to about 60, 65, 70, 75, 80, 85, 90 or 95% by weight of the formulation, i.e., hair cleansing formulation or hair conditioning formulation. In some examples, water may be present in an amount ranging from about 70% to about 90%, or about 75% to about 85%, or about 80% to about 90% by weight % of the formulation, i.e., hair cleansing formulation or hair conditioning formulation. In some examples, water may be present in an amount ranging from about 75% to about 85% in the hair cleansing formulation and from about 80% to about 90% in the hair conditioning formulation, by weight % of the respective formulations.

As used herein, the term “active materials” means the chemical entities providing a technical effect, for example a surfactant molecule, other than water. Most of the raw materials purchased from suppliers in liquid form are in an aqueous solution or suspension. When specified, and to avoid any confusion, the present disclosure refers to the weight % of active molecules, surfactant molecules for example, therefore specifically excluding the water content of the raw materials, for example surfactants, for the determination of the weight % of ingredient present in the formulations, i.e., hair cleansing formulation or hair conditioning formulation, according to the disclosure.

Active materials present in the surfactant mixture may amount for at least 11%, in weight %, of the cleansing formulation. In some examples, the active materials present in the surfactant mixture may amount for at least 12%, in weight %, of the cleansing formulation. In some examples, the active materials present in the surfactant mixture may amount for less than 15%, in weight %, of the cleansing formulation. In some examples, the active materials present in the surfactant mixture may amount for less than 14%, in weight %, of the cleansing formulation. In some examples, the active materials present in the surfactant mixture may amount for less than 13%, in weight %, of the cleansing formulation.

In one or more examples of the system for hair care, the hair cleansing formulation may further comprise a superhydrophilic amphiphilic copolymer.

In one or more examples of the system for hair care, the superhydrophilic amphiphilic copolymer may comprise a starch-based polysaccharide derived from potato or tapioca modified with dodecenyl succinic anhydride. Preferably the superhydrophilic amphiphilic copolymer may be a Sodium Hydrolyzed Potato Starch Dodecenylsuccinate.

In some examples, the superhydrophilic amphiphilic copolymer may be present in an amount ranging from 0.2% to 1.5% of the hair cleansing formulation, in weight %; or from 0.3% to 1%, or from 0.4% to 0.5%. Wherein the weight % refers to superhydrophilic amphiphilic copolymer active material content, in other words, excluding the water content of the superhydrophilic amphiphilic copolymer raw material.

As referenced herein, the terms “a starch-based polysaccharide derived from potato or tapioca modified with dodecenyl succinic anhydride” means a polysaccharide based on starch, i.e., derived from potato or tapioca, wherein said polysaccharide has been modified by esterification with dodecenyl succinic anhydride to convert some of the hydrophilic units of the polysaccharide into amphiphilic units, to provide the said starch-based polysaccharide derived from potato or tapioca modified with dodecenyl succinic anhydride. This starch-based polysaccharide derived from potato or tapioca modified with dodecenyl succinic anhydride may be added to a carrier, preferably a cosmetically-acceptable carrier.

In one or more examples of the system for hair care, the ratio of active material from the superhydrophilic amphiphilic copolymer relative to the active materials present in the surfactant mixture may be from 1:20 to 1:35.

In some examples, the ratio of active material from the superhydrophilic amphiphilic copolymer relative to the active materials present in the surfactant mixture may be from 1:25 to 1:30, or from 1:27 to 1:30, or from 1:28 to 1:29.

In some examples, the hair cleansing formulation may further comprise at least one cationic polymer. In some examples, the cationic polymer may be selected among polymers of Hydroxypropyltrimonium; Polyquaternium such as Polyquaternium-5, Polyquaternium-6, Polyquaternium-7, Polyquaternium-11, Polyquaternium-14, Polyquaternium-15, Polyquaternium-28, Polyquaternium-39, Polyquaternium-44; Polyquaternium-76; silicones and silicone derivatives such as cationically modified dimethicone polymers or copolymers. In some examples, the cationic polymer may be selected among Guar Hydroxypropyltrimonium Chloride, Starch Hydroxypropyltrimonium Chloride, Hydroxypropyl Guar Hydroxypropyltrimonium Chloride, or mixture thereof.

In some examples, the active material amount of cationic polymer present in the hair cleansing formulation may be from 0.1% to 1%, in weight % of the hair cleansing formulation.

In some examples, in the hair cleansing formulation, the active materials ratio of cationic polymer to surfactant mixture may be from 1:20 to 1:80.

In some examples, in the hair cleansing formulation, the active materials ratio of cationic polymer relative to the superhydrophilic amphiphilic copolymer may be from 1:0.9 to 1:2.5.

In some examples, in the hair cleansing formulation, a ratio of the active materials between the amphoteric surfactant, the anionic surfactant and the non-ionic surfactant may be in the range (2-3):(0.5-1.5):(0.5-1.5).

In some examples, in the hair cleansing formulation, the amphoteric surfactant may be an amphocarboxylates selected among mono or di alkylamphoacetates; an alkylbetaines; an amidoalkyl betaines; an amidoalkyl sultaines; an amphophosphates; a phosphorylated imidazolines selected among phosphobetaines and pyrophosphobetaines; a carboxyalkyl alkyl polyamines; an alkylimino-dipropionates; a mono or di alkylamphoglycinates; a mono or di alkylamphoproprionates; a N-alkyl β-aminoproprionic acids; an alkylpolyamino carboxylates; and mixtures thereof. In some examples, the amphoteric surfactant is selected among amidoalkyl betaines such as cocamidopropylbetaine.

In some examples, in the hair cleansing formulation, the amphoteric surfactant may be present in an amount ranging from 10% to 40% of the surfactant mixture active materials, in weight %. In some examples, the amphoteric surfactant is present in an amount ranging from 20% to 30% of the surfactant mixture active materials, in weight %; or from 20% to 25%, or from 22% to 24%.

In some examples, in the hair cleansing formulation, the anionic surfactant in the hair cleansing formulation may be selected among: alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl laurates, fatty alkyl sulfoacetates, alkyl phosphates, and mixtures of two or more thereof. In some examples, the anionic surfactant is selected among fatty acyl amino acids such as fatty acyl taurate. For example, in the hair cleansing formulation, the anionic surfactant may be Sodium Methyl Cocoyl Taurate.

In some examples, in the hair cleansing formulation, the anionic surfactant may be present in an amount ranging from 40% to 75% of the surfactant mixture active materials, in weight %. In some examples, in the hair cleansing formulation, the anionic surfactant may be present in an amount ranging from 40% to 65%, of the surfactant mixture active materials, in weight %; or from 45% to 60%, or from 50% to 55%, or from 51% to 52%

In some examples, the non-ionic surfactant in the hair cleansing formulation may be selected among fatty alcohol acid or amide ethoxylates, monoglyceride ethoxylates, sorbitan ester ethoxylates, long chain alkyl glucosides or polyglucosides, and mixtures thereof.

As referenced herein, the term “Long chain alkyl glucosides or polyglucosides” means the condensation products of (a) a long chain alcohol containing from about 6 to about 22, and preferably from about 8 to about 14 carbon atoms, with (b) glucose or a glucose-containing polymer.

In some examples, the non-ionic surfactant present in the hair cleansing formulation is selected among alkyl glucosides comprising from about 1 to about 6 glucose residues per molecule of alkyl glucoside. In one example, the glucoside is decyl glucoside. For example, the non-ionic surfactant present in the hair cleansing formulation may be decyl glucoside.

Non-ionic surfactants such as alkyl glucosides provide particular benefits in hair cleansing formulations because they are mild to the skin and eyes and are derived from natural ingredients, and therefore are renewable. Mildness is defined herein as the ability of a product to be applied to the skin or hair of a user with a low or negligible level of irritation. Skin and/or eye mildness of the composition of the disclosure may be determined using one or more of tests such as EpiDerm™ Skin Model (MatTek Corporation, Ashland, MA), Viability Assay, IL-1α Immunoassay, and EpiOcular™ Human Cell Construct Kit (MatTek Corporation, Ashland, MA) described in WO2018/204752.

In some examples, in the hair cleansing formulation, the non-ionic surfactant may be present in an amount ranging from 9% to 35% of the surfactant mixture active materials, in weight %. Preferably the non-ionic surfactant may be present in an amount ranging from 10% to 30%, or from 20% to 30%, or from 23% to 28%, or from 25% to 28%.

In one example, in the hair cleansing formulation, the surfactant mixture may comprise: from 40% to 70%, of the anionic surfactant, from 10% to 40% of the amphoteric surfactant, and from 9% to 35%, of the non-ionic surfactant; expressed in weight % of the surfactant mixture. The proportion above relates to active materials present in the surfactant mixture.

In another example, in the hair cleansing formulation, the surfactant mixture may comprise: from 40% to 65% of the anionic surfactant, from 20% to 30% of the amphoteric surfactant, and from 10% to 30% of the non-ionic surfactant; expressed in weight % of the surfactant mixture; or from 45% to 60% of the anionic surfactant, from 20% to 30% of the amphoteric surfactant, and from 20% to 30% of the non-ionic surfactant, or from 50% to 55% of the anionic surfactant, from 20% to 25% of the amphoteric surfactant, and from 23% to 28% of the non-ionic surfactant or from 51% to 52% of the anionic surfactant, from 22% to 24% of the amphoteric surfactant, and from 25% to 26% of the non-ionic surfactant; wherein all % are expressed in weight % of the surfactant mixture and relate to active materials present in the surfactant mixture.

In some examples, the hair cleansing formulation further comprises at least one hair conditioner. The hair conditioner may be selected among cationic surfactants having a conditioning effect, such as Cetrimonium Chloride, Stearamidopropyl Dimethylamine, Distearyldimonium Chloride, Lauryl Methyl Gluceth-10 Hydroxypropyldimonium Chloride); cationic polymers having a conditioning effect, such as cationically-modified polysaccharides, including Polyquaternium-10, Polyquaternium-24, Polyquaternium-67, and cationic polymers derived from the (co) polymerization of ethylenically-unsaturated cationic monomers with optional hydrophilic monomers, including Polyquaternium-5, Polyquaternium-6, Polyquaternium-7, Polyquaternium-11, Polyquaternium-14, Polyquaternium-15, Polyquaternium-28, Polyquaternium-39, Polyquaternium-44; Polyquaternium-76; silicones and silicone derivatives, such as Dimethicone, alkylmethicone, cetyldimethicone, cyclomethiconedimethicone, laurylmethicone, octyldimethicone ethoxyglucoside and other alkyl-, polyalkyloxy-, cationically-, anionically-modified dimethicone polymers or copolymers; and emollients having a conditioning effect such as Caprylic/Capric Triglycerides, Mineral Oil, Petrolatum, Di-PPG-2 Myreth-10 Adipate, Isopropyl Palmitate, lanolin, petrolatum, paraffin, beeswax, squalene, plant oils, such as coconut, jojoba, sesame, almond, and cottonseed, cetyl alcohol, olive oil (oleic acid), triethylhexanoin, shea butter, isopropyl myristate, C12-C15 alkyl, cocoglycerides and cocoa butter; essential oils and carrier oils having a conditioning effect for example Sea Buckthorn seed oil, the INCI name being Hippohae rhamnioides seed oil, the seed oil (Punica granatum seed oil), the Argan oil, the INCI name being Argania spinosa nut oil, the Camellia seed oil, the INCI name being Camellia oleifer seed oil, the Rosemary essential oil, the INCI name being Rosmarinu officinalis, the Tea Tree oil, the INCI name being Melaleuca alternifolia Leaf Oil, Lecithin, the INCI name being Lecithin, Helichrysum oil, the INCI name being Helichrysum italicum oil, Meadowfoam seed oil, the NCI name being Limnanthes alba seed oil, the Coffee Arabica oil, the INCI name being coffee arabica oil, and the d-alpha tocopherol is commonly known as Vitamin E.

In some examples, the hair cleansing formulation comprises at least one hair conditioner selected among: PEG-7 Amodimethicone, Amodimethicone, Polyquaternium-6, Coco-Glucoside (and) Glyceryl Oleate, and combinations thereof.

In some examples, the hair cleansing formulation comprises a cationic polymer having hair conditioning properties. In these examples, the cationic polymer is different from a hair conditioner that may be present in the hair cleansing formulation.

As described herein, the hair cleansing formulation according to the present disclosure system for hair care includes a cationic polymer and may also comprise a hair conditioner. Where both the cationic polymer and the hair conditioner are present simultaneously in the hair cleansing formulation their chemical structures are different. In other words, the cationic polymer of the hair cleansing formulation is not the hair conditioner of the hair cleansing formulation.

In one or more examples, the hair conditioning formulation further comprises at least one humectant. For example, the humectant present in hair conditioning formulation may be glycerin. In some examples, the humectant may be present in the hair conditioning formulation in an amount ranging from 0.5% to 2.5%, or 1% to 2%, in weight % of the hair conditioning formulation.

In one or more examples, the hair conditioning formulation further comprises at least one emulsion stabilizer. In some examples, the emulsion stabilizer present in the hair conditioning formulation is cetearyl alcohol. In some examples, the emulsion stabilizer is present in the hair conditioning formulation in an amount ranging from 3% to 6%, or 4% to 4.5% in weight % of the hair conditioning formulation.

In one or more examples, the at least one conditioning agent present in the hair conditioning formulation may be selected among Behentrimonium chloride, Stearamidopropyl Dimethylamine and dimethicone, or mixtures thereof. In some examples, the at least one conditioning agent is present in the hair conditioning formulation in an amount ranging from 2 to 5%, preferably 2.5 to 4%, preferably 3 to 3.5%, in weight %, of the hair conditioning formulation.

In one or more examples, the at least one emollient present in the hair conditioning formulation is selected among Isopropyl myristate and Coco caprylate/caprate, or mixtures thereof. In some examples, the at least one emollient is present in the hair conditioning formulation in an amount ranging from 0.1 to 2%, preferably 0.5 to 1%, in weight %, of the hair conditioning formulation.

In one or more examples, the at least one non-ionic surfactant present in the hair conditioning formulation is selected among alkoxylated alcohols, ethoxylated alcohols or combination thereof. In some examples, the at least one non-ionic surfactant is selected among Steareth-20, polysorbate-20, Ceteareth-20, Steareth-10 or other C16-C18 ethoxylated fatty alcohols with a number of ethoxylation from 10 to 30, or combinations thereof. In some examples, the at least one non-ionic surfactant may be present in the hair conditioning formulation in an amount ranging from 0.01 to 1%, preferably 0.1 to 0.5%, in weight %, of the hair conditioning formulation.

In one or more examples of the system for hair care, the hair cleansing formulation comprises a surfactant mixture, wherein the surfactant mixture comprises at least 20 to 25%, of the amphoteric surfactant, wherein the amphoteric surfactant may be an amidoalkyl betaines, 50 to 55% of the anionic surfactant, wherein the anionic surfactant may be a fatty acyl taurate, and 23 to 28% of the non-ionic surfactant, wherein the non-ionic surfactant may be an alkyl glucoside; a starch-based polysaccharide derived from potato or tapioca modified with dodecenyl succinic anhydride; at least one cationic polymer selected from Guar Hydroxypropyltrimonium Chloride, Starch Hydroxypropyltrimonium Chloride, Hydroxypropyl Guar Hydroxypropyltrimonium Chloride or mixture thereof; and water; wherein the active materials present in the surfactant mixture amount for at least 10%, preferably at least 11% or even preferably at least 12%, in weight %, of the hair cleansing formulation.

In one or more examples of the system for hair care, the hair cleansing formulation comprises a surfactant mixture, wherein the surfactant mixture comprises at least 20 to 25%, of the amphoteric surfactant, wherein the amphoteric surfactant may be cocamidopropylbetaine, 50 to 55% of the anionic surfactant, wherein the anionic surfactant may be sodium methyl cocoyl taurate, and 23 to 28% of the non-ionic surfactant, wherein the non-ionic surfactant may be decyl glucoside; a starch-based polysaccharide derived from potato or tapioca modified with dodecenyl succinic anhydride; at least one cationic polymer selected from Guar Hydroxypropyltrimonium Chloride, Starch Hydroxypropyltrimonium Chloride, Hydroxypropyl Guar Hydroxypropyltrimonium Chloride or mixture thereof; and water; wherein the active materials present in the surfactant mixture amount for at least 10%, preferably at least 11% or even preferably at least 12%, in weight %, of the hair cleansing formulation.

In one or more examples of the system for hair care, the hair conditioning formulation may further comprise: 2.5 to 4% of at least one conditioning agent wherein said conditioning agent may be selected among behentrimonium chloride, stearamidopropyl dimethylamine and dimethicone, or mixtures thereof, 0.5 to 1% of at least one emollient, wherein said emollient may be selected among Isopropyl myristate and Coco caprylate/caprate, or mixtures thereof, and 0.1 to 0.5% of at least one non-ionic surfactant, wherein said non-ionic surfactant may be selected among alkoxylated alcohols, ethoxylated alcohols or combination thereof.

In some examples, the non-ionic surfactant present in the hair conditioning formulation may be selected among: Steareth-20, polysorbate-20, Ceteareth-20, Steareth-10 or other C16-C18 ethoxylated fatty alcohols with a number of ethoxylation from 10 to 30, or combinations thereof.

In one or more examples of the system for hair care, the hair cleansing formulation may comprise the surfactant mixture including: 20 to 25%, of the amphoteric surfactant, wherein the amphoteric surfactant may be cocamidopropylbetaine, 50 to 55% of the anionic surfactant, wherein the anionic surfactant may be sodium methyl cocoyl laurate, and 23 to 28% of the non-ionic surfactant, wherein the non-ionic surfactant may be decyl glucoside; a starch-based polysaccharide derived from potato or tapioca modified with dodecenyl succinic anhydride; at least one cationic polymer selected from Guar Hydroxypropyltrimonium Chloride, Starch Hydroxypropyltrimonium Chloride, Hydroxypropyl Guar Hydroxypropyltrimonium Chloride or mixture thereof; and water; wherein the active materials present in the surfactant mixture amount for at least 10%, preferably at least 11% or even preferably at least 12%, in weight %, of the hair cleansing formulation. The hair conditioning formulation may include: 2.5 to 4% of at least one conditioning agent selected among behentrimonium chloride, stearamidopropyl dimethylamine and dimethicone, or mixtures thereof, 0.5 to 1% of at least one emollient selected among isopropyl myristate and coco caprylate/caprate, or mixtures thereof, and 0.1 to 0.5% of at least one nonionic surfactant selected among alkoxylated alcohols, ethoxylated alcohols or combination thereof.

In some examples, the non-ionic surfactant present in the hair conditioning formulation may be selected among: Steareth-20, polysorbate-20, Ceteareth-20, Steareth-10 or other C16-C18 ethoxylated fatty alcohols with a number of ethoxylation from 10 to 30, or combinations thereof.

Other examples of the present disclosure provide a method of manufacturing the hair cleansing formulation that may comprise the following steps: mixing the non-ionic surfactant and the amphoteric surfactant to an aqueous solution of the cationic polymer, to obtain a first solution, adding the anionic surfactant to said first solution and mix until homogenous to obtain a second solution, and adding an aqueous premix of superhydrophilic amphiphilic copolymer comprising from 1% to 10% water, to said second solution and mix until fully dissolved.

In one example, the method of manufacturing the hair cleansing formulation according to the system for hair care of the present disclosure comprises the following steps. mixing, at a temperature above 60° C., the non-ionic surfactant and the amphoteric surfactant to an aqueous solution of cationic polymer, to obtain a first solution, adding, at a temperature equal or below 60° C., the anionic surfactant to said first solution, optionally add a pearlizing agent, and mixing until homogenous to obtain a second solution, adding an aqueous premix of superhydrophilic amphiphilic copolymer comprising from 1% to 10% water, in weight %, to said second solution and mix until fully dissolved, to obtain a third solution, and adjusting the pH of the third solution between 4 and 5, optionally add a hair conditioner.

In another example, the present disclosure provides a method of treating human hair with the system for hair care as disclosed above. The method includes applying the hair cleansing formulation to the human hair, rinsing the hair cleansing formulation from the human hair; applying the hair conditioning formulation to the human hair, and rinsing the hair conditioning formulation from the human hair.

In another example, the method of treating human hair with the system for hair care according to the present disclosure is used in a cosmetic treatment method.

In another example, the present disclosure provides a method of reducing split ends in human hair. The method comprises contacting a subject hair with the system for hair care as described herein. In another example, the method of reducing split ends in human hair with the system for hair care according to the present disclosure is used in a cosmetic treatment method. In one or more examples, the method of reducing split ends in human hair with the system for hair care as described herein includes: applying the hair cleansing formulation to the human hair, rinsing the hair cleansing formulation from the human hair; applying the hair conditioning formulation to the human hair, and rinsing the hair conditioning formulation from the human hair.

A method for evaluating the ability to mend or reduce split ends was published by Wright and Szerszen (Ending the Cycle of Split Ends, Cosmetics & Toiletries, November 2011, p804). As disclosed, a moderate degree of partial closure of the hair split ends could be attributed to a 30% to 70% angle closure. A reasonable degree of partial closure could be attributed to an angle closure superior to 70%. Of course, complete closure of the hair split ends is the most desirable outcome and may also be achieved.

The method to reduce the split end of human hairs using the system for hair care according to the present disclosure may provide at least 80% of a split ends closure superior or equal to a moderate degree. In one example, the method to reduce the split end of human hairs using the system for hair care according to the present disclosure may provide at least 62% of complete split ends closure.

As referenced herein, the term “split ends closure superior or equal to a moderate degree” means that the angle closure of the hair split ends is of at least 30% but may be up to total closure.

In another example, the present disclosure provides a method of reducing breakage in human hair. The method comprises contacting a subject hair with the system for hair care as disclosed above. In one example, the method of reducing breakage in human hair with the system for hair care according to the present disclosure is used in a cosmetic treatment method. In some examples the method of reducing breakage in human hair with the system for hair care as described herein includes applying the hair cleansing formulation to the human hair, rinsing the hair cleansing formulation from the human hair; applying the hair conditioning formulation to the human hair, and rinsing the hair conditioning formulation from the human hair.

A method for evaluation hair breakage was published in the scientific literature by Evans & Park, A Statistical Analysis of Hair Breakage. II. Repeated Grooming Experiments, J. Cosmet. Sci., 61, 439-455, 2010. As disclosed, the method to reduce the breakage of human hairs using the system for hair care according to the present disclosure may provide at least a 65% reduction in hair breakage when compared to a control hair cleanser comprising 15%, in weight % of the control hair cleanser, of SLES (sodium lauryl ether sulfate) as surfactant.

For comparison, the cleansing formulation according to the system for hair care used alone (without the hair conditioner) provides a 29% reduction in hair breakage when compared to the control hair cleanser comprising 15%, in weight % of the control hair cleanser, of SLES as surfactant. It is highly beneficial to use a combination of the hair cleansing formulation and the hair conditioning formulation according to the present system for hair care to obtain a reduction in hair breakage.

In some examples, the system for hair care as described herein is used in a method to increase the volume of human hairs. In one example, the method of increasing the volume of human hair with the system for hair care according to the present disclosure is used in a cosmetic treatment method. The method to increase the volume of human hair using the system for hair care according to the present disclosure may provide at least a 64% increase in hair volume after application, and at least a 60% increase in hair volume 74 hours after application, when compared to a baseline control hair.

In some examples, the system for hair care according to the present disclosure is used in method to remove grease built up from human hairs. The method to remove grease build up from human hair according to the present disclosure may provide at least 91% of efficacy in grease removal, and preferably up to 93% of efficacy in grease removal.

In some examples, the hair system for hair care according to the present disclosure is used in a method to reduce the combing friction of human hairs. A reduced combing friction may be related to a benefit in detangling the hairs and facilitate the manageability of the hairs.

A method for evaluation of combing friction was published in the scientific literature by Garcia & Diaz (JSCC, 27, (1976) 379-398—Combability Measurements on Hair). The method to reduce the combing friction of human hairs using the system for hair care according to the present disclosure may provide at least a 77% reduction, preferably 80% reduction, in dry hair combing force when compared to the control hair cleanser comprising 15%, in weight % of the control hair cleanser, of SLES (sodium lauryl ether sulfate) as surfactant. The method to reduce the combing friction of human hairs using the system for hair care according to the present disclosure may provide at least a 93% reduction, in wet hair combing force when compared to the control hair cleanser comprising 15%, in weight % of the control hair cleanser, of SLES (sodium lauryl ether sulfate) as surfactant.

The present disclosure may be better understood through the following examples, which are provided as illustrations only and are not intended to be limiting to any specific combination of elements.

### Example 1

The following Compositions E1-E12 according to the disclosure and Comparative Compositions C1-C5 were made using the ingredients shown in the following Tables. Ingredients proportions are given in weight % of the composition total mass. For some ingredients, for example surfactants, the corresponding weight % of active material respective to the composition is also indicated (Active %).

TABLE 1A
Shampoo Composition

| | | Weight % |
|---|---|---|
| | | (Active %) |
| INCI Name | Function | E1 |
| Water | Vehicle | QS to 100 |
| Guar Hydroxypropyltrimonium Chloride | Conditioner | 0.20 |
| Sodium Hydrolyzed Potato Starch | Conditioner | 0.50 |
| Dodecenylsuccinate | | |
| Coco-Glucoside (and) | Conditioner | 1.00 |
| Hydrogenated Castor Oil | | |
| Amodimethicone | Conditioner | 0.20 |
| PEG-150 Distearate | Viscosity adjustor | Up to 1.00 |
| Hexylene Glycol | Viscosity adjustor | Up to 0.89 |
| Glycol Distearate | Pearlizer | 0.70 |
| Sodium Methyl Cocoyl Taurate | Surfactant | 26.50 (6.4) |
| Cocamidopropyl Betaine | Surfactant | 8.00 (2.9) |
| Decyl Glucoside | Surfactant | 6.00 (3.2) |
| Sodium Benzoate | Preservative | 0.40 |
| Citric Acid | pH adjustor | Up to 0.89 |
| Sodium Hydroxide | pH adjustor | Up to 0.89 |

Composition E1 was prepared as follows.

Water was added to a main pot and began heating to 70° C. under constant mixing. Guar Hydroxypropyltrimonium Chloride was added, then a small amount of Citric Acid was added and mixed until homogeneous. At temperature 55° C. or higher, PEG-150 Distearate was added and mixed until fully dissolved. After full dissolution of PEG-150 Distearate, Glycol Distearate was added and mixed until fully dissolved then turned off heat. Once the temperature reached between 60° C. and 70° C., Cocamidopropyl Betaine was added, then Decyl Glucoside was added. Next Sodium Methyl Cocoyl Taurate was added and mixed until fully dissolved and homogenous. Next, Sodium Hydrolyzed Potato Starch Dodecenylsuccinate Water premix (0.50 wt % Sodium Hydrolyzed Potato Starch Dodecenylsuccinate and 5.00 wt % Water) was added, then Sodium Benzoate was added and mixed until fully dissolved. The pH was adjusted to the target pH, between 4.20 and 4.60, with Citric Acid before Coco-Glucoside, Hydrogenated Castor Oil, and Amodimethicone were added. Once the temperature reached 40° C. or lower, the solution was mixed for at least 15 minutes. The pH and viscosity were tested and, if necessary, the pH was adjusted with Citric Acid or Sodium Hydroxide to the target pH between 4.20 and 4.60, Then, the viscosity was adjusted with Hexylene Glycol as needed to target a viscosity between 8,000-15,000 cps. Composition E1 exhibited pearlized appearance and demonstrated desirable texture, good foaming properties as determined by testing using at least one of the Ross-Miles or Sita-FoamTester testing methods, good conditioning properties, and desirable cleansing efficacy as determined by testing using, for example, a Sita-FoamTester to analyze characteristics of the composition.

TABLE 1B
Shampoo Composition

| | | Weight % |
|---|---|---|
| | | (Active %) |
| INCI Name | Function | E2 |
| Water | Vehicle | QS to 100 |
| Starch Hydroxypropyltrimonium | Conditioner | 1.50 |
| Chloride Hydrolyzed Potato | | |
| Starch | | |
| Sodium Hydrolyzed Potato Starch | Conditioner | 0.50 |
| Dodecenylsuccinate | | |
| Coco-Glucoside (and) Glyceryl Oleate | Conditioner | 0.50 |
| Amodimethicone | Conditioner | 0.20 |
| PEG-150 Distearate | Viscosity adjustor | Up to 1.00 |
| Hexylene Glycol | Viscosity adjustor | Up to 0.89 |
| Sodium Methyl Cocoyl Taurate | Surfactant | 26.50 (6.4) |
| Cocamidopropyl Betaine | Surfactant | 8.00 (2.9) |
| Decyl Glucoside | Surfactant | 6.00 (3.2) |
| Sodium Benzoate | Preservative | 0.40 |
| Citric Acid | pH adjustor | Up to 0.89 |
| Sodium Hydroxide | pH adjustor | Up to 0.89 |

Composition E2 was prepared as follows.

Water was added to the main pot and began heating to 70° C. under constant mixing. At temperature 55° C. or higher, PEG-150 Distearate was added and mixed until fully dissolved. The heat was then turned off. Once the temperature was reached between 60° C. and 70° C., Cocamidopropyl Betaine was added, then Decyl Glucoside was added. Next, Sodium Methyl Cocoyl Taurate was added and mixed until fully dissolved and homogenous. Next, Sodium Hydrolyzed Potato Starch Dodecenylsuccinate Water premix (0.50 wt % Sodium Hydrolyzed Potato Starch Dodecenylsuccinate and 5.00 wt % Water) was added, and then Sodium Benzoate was added and mixed until fully dissolved. The pH was adjusted to a target of 4.40 with 50% Citric Acid solution before adding Coco-Glucoside and Glyceryl Oleate and mixed until fully dissolved. Next, Amodimethicone and Starch Hydroxypropyltrimonium Chloride were added. Once the temperature reached 40° C., the solution was mixed for at least 15 minutes. The pH and viscosity were tested and, if necessary, the pH was adjusted with Citric Acid or Sodium Hydroxide to achieve a target pH, between 4.20 and 4.60. Then, viscosity was adjusted with Hexylene Glycol as needed to achieve target a viscosity between 7,000-13,000 cps. Composition E2 exhibited clear appearance. Further, the composition demonstrated desirable texture, good foaming properties, good conditioning properties, and desirable cleansing efficacy.

TABLE 1C
Additional Shampoo Compositions

| | | Weight % (Active %) | | | | |
|---|---|---|---|---|---|---|
| INCI Name | Function | E3 | E4 | E5 | E6 | E7 |
| Water | Vehicle | QS to | QS to | QS to | QS to | QS to |
| | | 100 | 100 | 100 | 100 | 100 |
| Guar | Conditioner | 0.20 | 0.20 | 0.20 | | |
| Hydroxypropyltrimonium | | | | | | |
| Chloride | | | | | | |
| Starch | Conditioner | | | | 1.50 | 1.50 |
| Hydroxypropyltrimonium | | | | | | |
| Chloride | | | | | | |
| Sodium Hydrolyzed Potato | Conditioner | 0.50 | 0.50 | 0.50 | 0.50 | 0.50 |
| Starch Dodecenylsuccinate | | | | | | |
| Coco-Glucoside (and) | Conditioner | 1.50 | 1.50 | | 0.5 | |
| Hydrogenated Castor Oil | | | | | | |
| Amodimethicone | Conditioner | | | | | 0.20 |
| PEG-150 Distearate | Viscosity | 0.25 | 0.20 | 0.50 | 0.50 | 0.50 |
| | adjustor | | | | | |
| Hexylene Glycol | Viscosity | Up to | Up to | | Up to | |
| | adjustor | 0.89 | 0.89 | | 0.89 | |
| Glycol Distearate | Pearlizer | 3.0 | 3.0 | 0.70 | | |
| Sodium Methyl Cocoyl | Surfactant | 24.00 | 30.60 | 30.00 | 26.5 | 30.00 |
| Taurate | | (7.2) | (9.2) | (7.2) | (6.4) | (7.2) |
| Cocamidopropyl Betaine | Surfactant | 9.00 | 9.00 | 8.00 | 8.00 | 8.00 |
| | | (3.2) | (3.2) | (2.9) | (2.9) | (2.9) |
| Decyl Glucoside | Surfactant | 4.00 | 4.00 | 3.00 | 6.00 | 4.00 |
| | | (2.1) | (2.1) | (1.6) | (3.2) | (2.1) |
| Sodium Benzoate | Preservative | 0.40 | 0.40 | 0.40 | 0.40 | 0.40 |
| Citric Acid | pH adjustor | Up to | Up to | 1.25 | Up to | 1.20 |
| | | 1.78 | 1.78 | | 0.89 | |
| Sodium Hydroxide | pH adjustor | Up to | Up to | Up to | Up to | Up to |
| | | 0.89 | 0.89 | 0.89 | 0.89 | 0.89 |

Compositions E3-E5 and E6-E7 were prepared in a similar process as E1 and E2, respectively, as described above.

TABLE 1D
Additional Shampoo Compositions

| | | Weight % (Active %) | | | | |
|---|---|---|---|---|---|---|
| INCI Name | Function | E8 | E9 | E10 | E11 | E12 |
| Water | Vehicle | QS to | QS to | QS to | QS to | QS to |
| | | 100 | 100 | 100 | 100 | 100 |
| Guar | Conditioner | 0.20 | 0.20 | 0.20 | | |
| Hydroxypropyltrimonium | | | | | | |
| Chloride | | | | | | |
| Starch | Conditioner | | | | 1.50 | 1.00 |
| Hydroxypropyltrimonium | | | | | | |
| Chloride | | | | | | |
| Sodium Hydrolyzed Potato | Conditioner | 0.50 | 0.50 | 0.50 | 0.50 | 0.50 |
| Starch Dodecenylsuccinate | | | | | | |
| Coco-Glucoside (and) | Conditioner | 1.50 | 1.50 | 1.50 | | |
| Hydrogenated Castor Oil | | | | | | |
| PEG-150 Distearate | Viscosity | 0.50 | 0.30 | 0.40 | 0.80 | 0.60 |
| | adjustor | | | | | |
| Hexylene Glycol | Viscosity | | Up to | | Up to | |
| | adjustor | | 0.89 | | 0.89 | |
| Glycol Distearate | Pearlizer | | 0.6 | | | |
| Sodium Methyl Cocoyl | Surfactant | | 30.00 | 35.94 | 30.00 | |
| Taurate | | | (7.2) | (8.6) | (7.2) | |
| Sodium Lauroyl Methyl | Surfactant | 29.96 | | | | 29.96 |
| Isethionate | | (10.04) | | | | (10.04) |
| Cocamidopropyl Betaine | Surfactant | 4.86 | 9.00 | 4.86 | 8.00 | 4.86 |
| | | (1.7) | (3.2) | (1.7) | (2.9) | (1.7) |
| Decyl Glucoside | Surfactant | 2.17 | 2.00 | 2.17 | 2.00 | 2.17 |
| | | (1.2) | (1.1) | (1.2) | (1.1) | (1.2) |
| Sodium Benzoate | Preservative | 0.40 | 0.40 | 0.40 | 0.40 | 0.40 |
| Citric Acid | pH adjustor | 1.23 | 1.00 | 1.23 | 1.25 | 1.23 |

Compositions E8-E10 and E11-E12 were prepared by the same process as E1 and E2, respectively, as described above. Sodium Lauroyl Methyl Isethionate was used in E8 and E12 while Sodium Methyl Cocoyl Taurate was added in E9, E10, and E11.

TABLE 2
Conditioner Composition

| | | Weight % |
|---|---|---|
| | | (Active %) |
| INCI Name | Function | E13 |
| Water | Vehicle | QS to 100 |
| Glycerin | Conditioner | 1.50 |
| Dimethicone (350cs) | Conditioner | 0.75 |
| Coco-Caprylate/Caprate | Conditioner | 0.25 |
| Isopropyl Myristate | Conditioner | 0.50 |
| Stearamidopropyl Dimethylamine | Conditioner | 0.50 |
| Behentrimonium Chloride | Surfactant | 2.50 |
| Steareth-20 | Surfactant | 0.30 |
| Cetearyl Alcohol | Rheology Modifier | 4.20 |
| Sodium benzoate | Preservative | 0.50 |
| Citric acid | pH adjustor | Up to 0.89 |
| Sodium Hydroxide | pH adjustor | Up to 0.89 |

Inventive Example E13 was prepared in one of two ways: a Two Pot Process or a One Pot Process.

The Two Pot Process is performed as follows:

Under constant mixing, the water began heating in a main pot at between 65° C. and 75° C. While heating, Glycerin was added as heating continued. Once the temperature reached 60° C. or higher, Behentrimonium Chloride was added. Meanwhile, Coco-Caprylate/Caprate, Isopropyl Myristate, Stearamidopropyl Dimethylamine, Steareth-20, and Cetearyl Alcohol were added into a second pot and began to be heated to between 65° C. and 75° C. Once the temperature of both the main pot and second pot reached 65° C. or above, the contents of the second pot were added to the main pot and mixed for a minimum of 20 minutes while maintaining a temperature between 65° C. and 75° C. The heat was turned off and Sodium Benzoate was added and mixed until fully dissolved. Once the temperature reached 60° C. or below, the remaining Water and Dimethicone (350 cs) were slowly added. Once the temperature reached 40° C., the solution was mixed for at least 15 minutes. The pH was tested and, if necessary, the pH was adjusted with Citric Acid or Sodium Hydroxide to a target pH of between 4.10 and 4.50. In some examples, Glycerin may be added any point after Sodium Benzoate.

The One Pot Process is performed as follows:

Under constant mixing, the water began being heated to 70° C. While heating, Glycerin was added and as heating continued. Once the temperature reached 60° C. or higher, Behentrimonium Chloride was added. Once the temperature reached 65° C. or higher, the following ingredients were added one at a time, and mixed until completely dissolved between each addition: Cetearyl Alcohol, Stearamidopropyl Dimethylamine, Steareth-20, Isopropyl Myristate, and Coco-Caprylate/Caprate. After the Coco-Caprylate/Caprate was added, the temperature was maintained between 65° C. to 70° C. and mixed for 20 minutes. The heat was turned off and Sodium Benzoate was added and mixed until fully dissolved. Once the temperature reached 60° C. or lower, the remaining water and Dimethicone (350 cs) were added. Once the temperature reached 40° C., the solution was mixed for at least 15 minutes. The pH was tested and, if necessary, the pH was adjusted with Citric Acid or Sodium Hydroxide to a target pH of between 4.10 and 4.50. In some examples, Glycerin may be added any point after Sodium Benzoate.

Composition E13 exhibited smooth texture and an opaque appearance. Further, the composition demonstrated good conditioning properties.

TABLE 3
Comparative Examples

| | | Weight % (Active %) | | | | |
|---|---|---|---|---|---|---|
| INCI Name | Function | C1 | C2 | C3 | C4 | C5 |
| Water | Vehicle | QS to | QS to | QS to | QS to | QS to |
| | | 100 | 100 | 100 | 100 | 100 |
| Guar | Conditioner | 0.20 | 0.20 | | 0.25 | 0.25 |
| Hydroxypropyltrimonium | | | | | | |
| Chloride | | | | | | |
| Starch | Conditioner | | | 1.00 | | |
| Hydroxypropyltrimonium | | | | | | |
| Chloride | | | | | | |
| Sodium Hydrolyzed Potato | Conditioner | 0.50 | 0.50 | 0.50 | 0.50 | 0.50 |
| Starch Dodecenylsuccinate | | | | | | |
| Coco-Glucoside (and) | Conditioner | 1.50 | 1.50 | | 2.0 | 2.0 |
| Hydrogenated Castor Oil | | | | | | |
| Amodimethicone | Conditioner | | | | | |
| PEG-150 Distearate | Viscosity | 0.70 | 1.00 | 0.20 | up to | up to |
| | adjustor | | | | 1.00 | 1.00 |
| Hexylene Glycol | Viscosity | | | | Up to | Up to |
| | adjustor | | | | 0.89 | 0.89 |
| Glycol Distearate | Pearlizer | | | | | |
| Sodium Methyl Cocoyl | Surfactant | 28.13 | 28.00 | 28.13 | 24.00 | 24.00 |
| Taurate | | (6.8) | (6.7) | (6.8) | (7.2) | (7.2) |
| Cocamidopropyl Betaine | Surfactant | 3.80 | 3.80 | 3.79 | 14.00 | |
| | | (1.4) | (1.4) | (1.4) | (5.0) | |
| Coco-Betaine | Surfactant | | | | | 17.00 |
| | | | | | | (5.00) |
| Decyl Glucoside | Surfactant | 1.70 | 1.69 | 1.70 | | |
| | | (0.9) | (0.9) | (0.9) | | |
| Sodium Benzoate | Preservative | 0.40 | 0.40 | 0.40 | 0.40 | 0.40 |
| Citric Acid | pH adjustor | 1.23 | 1.23 | 1.23 | Up to | Up to |
| | | | | | 1.78 | 1.78 |
| Sodium Hydroxide | pH adjustor | | | | 0.89 | 0.89 |

Comparative Examples C1-C5 were prepared in the same process as E1 and E2, as described above, where C1, C2, C4, and C5 match the process for E1 and C3 matches the process for E2. Coco-Betaine was added in C5 in the process where Cocamidopropyl Betaine would be added for C1-C4. Comparative examples C1 through C5 did not exhibit the desired foaming properties nor cleansing efficacy.

### Example 2

### Example 2A. Split End Closure Evaluation

Composition E1 Shampoo and Combination of E1 Shampoo and E13 Conditioner were evaluated for their split end closure properties on fibers treated with such compositions. Fifty (50) European Medium Brown hair tresses were first damaged and bleached using a 6% hydrogen peroxide solution at pH of 10.2 and left in contact with the bleach solution for 40 minutes under controlled temperature (40° C.), followed by repeated brushing to induce split ends which were randomly selected and individually labeled so they can be tracked. Each split end fiber substrate (50 for each composition) was imaged using a 25× stereo microscope before treatment with the compositions: applying shampoo composition E1 at 10% by weight of the tress (0.1 ml/g of hair) or shampoo and conditioner compositions E1 and E13 combo at 15% by weight of the tress (0.15 ml/g of hair) on damp hair, massaging for 30 sec and rinsing for 30 sec. All treatment was performed with an intellifaucet set at 40° C. with a controlled flow rate of 1 GPM. The treated hair fiber substrates were then air-dried over-night before taking post-treatment split end images.

### Split End Closure Evaluation:

Methods for evaluating the ability to mend split ends are published by Wright and Szerszen (Ending the Cycle of Split Ends, Cosmetics & Toiletries, November 2011, p804), and Rigoletto et al. (Semi-permanent split end mending with a polyelectrolyte complex, J. Cosmet. Sci., 58, 451-476, 2007; Polyelectrolyte complex for mending damaged hair, Cosmetics & Toiletries, 124 (3) 2009). The evaluation of the ability of the supplied regimens to induce split end closure was developed based on the 2011 procedure. Further, a grading scale to characterize the level of closure was introduced allowing for an evaluation of the durability, adopting a 5-point grading scale for evaluating the degree to which the split end has been closed. This grading scale shown in Table 4 allows for a more rapid yet accurate assessment of the state of the hair.

TABLE 4
Grading Scale

| | |
|---|---|
| 1 | No Closure |
| 2 | Minimal partial closure ~less than 30% angle closure |
| 3 | Moderate degree of partial closure ~between |
| | 30%-70% angle closure |
| 4 | Reasonable degree of partial closure ~more |
| | than70% angle closure |
| 5 | Complete closure |

The percentage of split ends closed of Compositions E1 and E1+E13 were tested using the test method set forth above. The results are shown in Table 5.

TABLE 5
Split ends closure after treatment with Shampoo and Conditioner Composition Treatment

| | Total # | # of fibers | # of fibers | % of fibers | % of fibers | Total % of |
|---|---|---|---|---|---|---|
| Treatment | of | partially | completely | partially | completely | fibers closed |
| Composition | fibers | closed (3, 4) | closed (5) | closed (3, 4) | closed (5) | (3, 4, and 5) |
| E1 | 50 | 21 | 22 | 42% | 44% | 86% |
| E1 + E13 | 50 | 9 | 31 | 18% | 62% | 80% |

The data in Table 5 illustrates that split ends treated with the composition E1 result in a total of 86% split end closure (>30% reduction in angle) after application, out of which 44% of split ends are completely closed. The date in Table 5 further illustrates that split ends treated with the compositions E1+E13 result in a total of 80% split end closure (>30% reduction in angle) after application, of which 62% of split ends are completely closed.

### Example 2B. Protection Against Breakage

Composition E1 Shampoo and Combination of E1 Shampoo and E13 Conditioner were evaluated for their properties of protecting against breakage via repeated grooming on fibers treated with such compositions.

Ten (10) 3 g, 8″ long, 1″ wide European medium brown tresses were bleached using a 9% hydrogen peroxide solution at pH of 10.2. The tresses were left in contact with the bleach solution for 20 minutes under controlled temperature conditions (40° C.). At the end of this process, tresses were thoroughly rinsed under an intellifaucet set at 40° C. with a controlled flow rate of 1.0 GPM. The bleaching procedure was repeated two more times. Treating the hair tresses with shampoo (E1) shampoo+conditions combo (E1+E13) and control solutions (15% SLES), respectively, which was performed with an intellifacucet set at 40° C. with a controlled flow rate of 1 GPM. All tresses were standardized with SLES prior to treatment. For 15% SLES and E1, applied SLES or E1 at 10% by weight of the tress (0.1 ml/g of hair) on damp hair, massaged 30 seconds, rinsed 30 seconds, squeezed out excess water, allowing hair to air dry and equilibrate at 60±2% RH and 22±2° C. prior to testing. For E1+E13, applied shampoo E1 on damp hair at 10% by weight of the tress (0.1 ml/g of hair), massaged for 30 sec and rinsed for 30 sec. Then, applied conditioner E13 on the damp hair at 15% by weight of the tress (0.15 ml/g of hair), massaged for 30 sec, and rinsed for 30 sec, allowing hair to air dry and equilibrate at 60±2% RH and 22±2° C. prior to testing.

After tresses were treated as outlined above, they were groomed using an automatic groomer for a total of 2,000 cycles, fibers were collected and assessed after every 200 cycles.

A method to determine whether a treatment will protect hair against breakage is provided by repeated grooming experiments. (Evans & Park, A Statistical Analysis of Hair Breakage. II. Repeated Grooming Experiments, J. Cosmet. Sci., 61, 439-455, 2010). In this test, the number of broken fibers is recorded as a function of repeated combing/brushing strokes. Any treatment that reduces snags, entanglements and abrasion can help in substantially lowering the number of broken fibers. Our methodology utilizes a custom-built automated grooming device which consists of a hollow rotating drum-like assembly, where four outer crossbars contain holders for mounting combs or brushes. The outer arms are detachable to allow for different holders to be mounted and experiments to be performed using a variety of combs or brushes. The four combs or brushes are mounted at 90° angles, allowing one complete drum revolution to comb (or brush) a tress four times. This entire set-up is replicated ten times in the horizontal direction allowing ten tresses to be combed simultaneously. Collection plates are located under each tress to save broken fiber fragments, while spacer plates on the rotating drum prevent cross contamination.

All experiments, in eight replicates per composition, were performed after overnight equilibration of the hair at 60% RH at 22° C.

The number of broken fibers of Compositions E1 and E1+E13 and control were tested using the Test Method set forth above. The results are shown in Table 6.

TABLE 6
Repeated Grooming Results

| | | Number of | Reduction |
|---|---|---|---|
| Treatment | Total # of | Broken Fibers | in |
| Composition | tresses | After 2,000 Strokes | breakage* |
| Control (15% | 10 | 55 | |
| SLES) | | | |
| Composition | 10 | 39 | 29% |
| E1 | | | |
| Composition | 10 | 19 | 65% |
| E1 + E12 | | | |

$$* \% Reduction in breakage = ( 1 - Breakage of treatment / Breakage of control ) \times 100.$$

As illustrated in Table 6, there is a statistical reduction in breakage for shampoo composition E1 and Shampoo Conditioner combination composition (E1+E13) compared with the Control (15% SLES). Particularly, E1 shows a reduction in breakage of 29% and E1+E13 shows reduction in breakage of 65% compared with the control.

### Example 2C. Build-Up Removal Testing

E1 Shampoo, E2 Shampoo, and Combination of E2 Shampoo and E13 Conditioner were evaluated for their silicone build-up removal properties on fibers treated with such compositions.

### Hair Tresses Pretreatment and Silicone Deposition.

5 Caucasian medium brown hair tresses supplied by International Hair Importers tested for each composition. Each tress was 8 inches long, 1 inch wide, and weighed approximately 3 g. The tresses were standardized with 0.15 ml of non-conditioning shampoo, massaged, and rinsed under intellifaucet water for 30 seconds each. The residue build-up was introduced by applying 0.45 g of silicone on virgin hair tresses to mimic greasy and dirty hair. The tresses were massaged manually and brushed for even distribution of the silicone along the hair fibers then blown dried on low heat for 5 minutes. This process was repeated for a total of 3 cycles. The tresses were scanned by ATR-FTIR spectroscopy to assess the buildup silicone content after 3× application.

### Cleansing and Conditioning Composition Application:

Regimen 1: Composition E1 equal to 10% weight of the tress was applied on the wet tresses for 30 seconds, then rinsed for 30 seconds.

Regimen 2: Composition E2 equal to 10% weight of the tress was applied on the wet tresses for 30 seconds, then rinsed for 30 seconds.

Regimen 3: Composition E2 equal to 10% weight of the tress was applied on the wet tresses for 30 seconds, and then rinsed for 30 seconds. Composition E13 equal to 15% weight of the tress was applied on the wet tresses for 30 seconds, and then rinsed for 30 seconds. After the tresses were dried, they were scanned by ATR-FTIR spectroscopy to evaluate the silicone removal.

### FTIR Spectroscopy Measurement of Build-Up Removal:

The FTIR data were recorded with a spotlight system 400 from PerkinElmer with an ATR accessory. The spectra were recorded with the following spectral parameters: Spectral resolution 8 cm⁻¹, 8 Scans accumulations, and range 4000-650 cm⁻¹.

5 hair tresses were used to evaluate the effective buildup removal of each regimen treatment outlined above. Several ATR-FTIR spectra were recorded on each hair tress.

6 FTIR spectra were recorded before the application of the compositions. 12 FTIR spectra were recorded after 3× silicone application. 12 FTIR spectra were recorded after each regimen treatment.

All the ATR-FTIR spectra were baseline corrected and normalized on the Amide I band, which is correlated to the protein content in the hair. The bands at 1260 and 1012 cm⁻¹ were used to evaluate the reduction of the build-up (silicone) content on the hair tresses after application of the shampoo and conditioner compositions.

TABLE 7
Efficacy of Silicone Build-up Removal

| | Total | Silicone marker 1260 | Silicone marker 1012 |
|---|---|---|---|
| Treatment | # of | removal after treatment | removal after treatment |
| Composition | tresses | application | application |
| Composition E1 | 5 | 79.51% | 82.87% |
| Composition E2 | 5 | 87.10% | 89.67% |
| Composition | 5 | 91.75% | 93.92% |
| E2 + E13 | | | |

As shown in Table 7, Compositions E1, E2 and E2+E13 exhibit efficacy at removing silicone build-up from the hair tresses. Composition E2+E13 was the most effective for silicone removal.

### Example 2D. Volume Measurement Via Image Analysis

E2 Shampoo and Combination of E2 Shampoo and E13 Conditioner were evaluated for their change in volume properties on fibers treated with such compositions. Eight (8) Round Medium Brown hair tresses (6 grams, 8 inches) per treatment group. All treatments were performed using an Intellifaucet set at 40° C. with a controlled flow rate of 1.0 GPM. All tresses were cleansed with SLES and leave overnight to dry before any treatments.

Baseline Sebum: Sebum is diluted with water in 1:10 ratios, 1 part of sebum is mixed with 10 parts of water. 2 g is applied and worked in with mascara brush for 10 strokes, per tress.

### Hair Tresses Treatment:

Shampoo E2 on damp hair: apply shampoo at 10% by weight of the tress (0.1 ml/g of hair), massage 30 sec, rinse 30 sec.

Combination of E2 Shampoo and E13 Conditioner: Shampoo E2 on damp hair, apply shampoo at 10% by weight of the tress (0.1 ml/g of hair), massage 30 sec, rinse 30 sec. Then Conditioner E13 on damp hair, apply conditioner at 15% by weight of the tress (0.15 ml/g of hair), massage 30 sec, rinse 30 sec.

The analysis was completed to compare sebum treated baseline to initial, 8 hour, 24 hour, 48 hour, 74 hour time points. Testing involved the use of an image analysis method to track the changes in tress dimensions and volume before and after treatment with Shampoo E2 or Combination of E2 Shampoo and E13 Conditioner under climate-controlled conditions. Prior to all treatments, tresses were equilibrated under specified controlled conditions in a climate chamber. High quality photographic images were acquired to characterize the initial state of the tresses for baseline. After treatment with Shampoo E2 or Combination of E2 Shampoo and E13 Conditioner, these hair tresses were again maintained at standard temperature and RH until all samples have been prepared. Tresses were again exposed to standard controlled conditions and additional photographic images were taken at appropriate durations. The volume dimensions (or Area at X hours) were measured from the captured images and determined using custom software.

The hair tresses treated with E2 Shampoo and compared to sebum treated tresses (baseline) show a statistical increase in volume after each time point.

The hair tresses treated with E2 Shampoo+E13 Conditioner and compared to sebum treated tresses (baseline) show a statistical increase in volume after each time point.

% Increase in volume after X hours is calculated as below:

$$\% Increase in volume = [ ( Area at X hours - Area at Baseline ) / ( Area at Baseline ) ] * 100.$$

At t=0 Hour (Initial)

E2 Shampoo shows a 100% increase in volume after application.

E2 Shampoo+E13 Conditioner shows a 64% increase in volume after application.

At t=74 Hour

E2 Shampoo shows a 97% increase in volume 74 hours after application.

E2 Shampoo+E13 Conditioner shows a 60% increase in volume 74 hours after application.

### Example 2E Measurement of Reduction of Dry Combing Forces and Wet Combing Forces

Shampoo E1, E2, E6, E7, Combination of E1 (Shampoo) and E13 (Conditioner), and Combination E2 and E13, were evaluated for their dry combing forces and wet coming forces, respectively, treated with such compositions.

One of the technical functions of shampoo and conditioning products is to lubricate the hair surface; and, in doing so, facilitate manageability and provide detangling benefits and lower combing friction.

Eight European medium brown hair tress, 6% Bleached hair tresses, (3.0 g, 8″ length, 1″ wide) are used per treatment group. All treatment is performed using an Intellifaucet set at 40° C. with a controlled flow rate of 1.0 GPM. All tresses cleansed with 15% SLES and left overnight to dry before any treatments.

Shampoo treatment: on damp hair, apply shampoo at 10% by weight of the tress (0.1 ml/g of hair), massage 30 seconds, rinse 30 seconds.

Conditioner treatment: on damp hair, apply conditioner at 15% by weight of the tress (0.15 ml/g of hair), massage 30 seconds, rinse 30 seconds.

For dry combing tests: All tresses are allowed to dry and equilibrate overnight at 60% RH.

### Testing:

Frictional forces experiments were performed in accordance with the method proposed by Garcia & Diaz (JSCC, 27, (1976) 379-398—Combability Measurements on Hair). Tensile tester is used to measure frictional forces while a hair tress is pulled through a comb. Combing experiments are performed in the wet state after treatment or dry state after treatment. Six combing strokes are performed per tress, while eight replicate hair tresses are used per sample to ensure statistical relevance.

TABLE 8
Measurement of Dry Combing Forces and Wet Combing Forces

| | % Reduction in | % Reduction |
|---|---|---|
| | Dry Combing | in wet Combing |
| Treatment | force vs Control | force vs Control |
| Composition | 15% SLES | 15% SLES |
| Composition E1 | 39 | 70 |
| Composition E2 | 33 | 71 |
| Composition E6 | 24 | 63 |
| Composition E7 | 24 | 48 |
| Composition E1 + E13 | 80 | 93 |
| Composition E2 + E13 | 77 | 93 |

As shown in Table 8, all shampoo Compositions E1, E2, E6 and E7 and combination of shampoo and conditioner E1+E13 and E2+E13 exhibit efficacy at reduction in both dry and wet combing force. Combinations of shampoo and conditioner E1+E13 and E2+E13 are the most effective for reduction in both dry and wet combing force.

### Example 3. Hair Lipid Evaluation

Co Compositions E14 and additional comparative examples C6, C7, and C8 were evaluated for gentleness in hair treatment in order to evaluate and compare lipid removal from hair. For each sample, five (5) Caucasian medium brown hair tresses were used for evaluation. Each tress was eight inches long, one inch wide, and weighed approximately three grams. The tresses were standardized with 0.3 mL of non-conditioning shampoo, massaged, and rinsed under an Intellifaucet for 30 seconds each to prepare for evaluation. Six FTIR spectra were recorded prior to the application of the cleanser.

Table 9, below, includes the formulation for E14 as shown above in addition to Comparative Examples C6, C7, and C8. C6 comprises 15% by weight of Sodium Lauryl Ether Sulfate in water. As shown in Table 9, E14 includes Sodium Methyl Cocoyl Taurate as a primary surfactant (anionic), includes Decyl Glucoside and Cocamidopropyl Betaine as co-surfactants (amphoteric/cationic/non-ionic), and includes Cocos Nucifera (Coconut) Fruit Extract, Hydrolyzed Milk Protein, Sodium Hydrolyzed Potato Starch Dodecenylsuccinate, Amodimethicone, Guar Hydroxypropyltrimonium Chloride, and Hydrogenated Castor Oil as conditioning ingredients. C7 includes Sodium Methyl Cocoyl Taurate and Sodium Methyl 2-sulfolaurate as primary surfactants (anionic), includes Cocamidopropyl Betaine, Coco-glucoside, and PEG-7 Glyceryl Cocoate as co-surfactants (amphoteric/cationic/non-ionic), and includes Panthenol, Hydrogenated Castor Oil, Polyquaternium-10, Hydrolyzed Silk, and Nymphaea Alba Root Extract as conditioning ingredients. C8 includes Sodium C14-16 Olefin Sulfonate as a primary surfactant (anionic), includes Cocamidopropyl Betaine as a co-surfactant (amphoteric/cationic/non-ionic), and includes Curcuma Longa (Turmeric) Root Extract, Hydrolyzed Collagen, Dimethicone, Guar Hydroxypropyltrimonium, Glycerin, Cocos Nucifera Oil, Hydrogenated Castor Oil, and Polyquaternium-10 as conditioning ingredients.

The Applicant notes that C7 includes hydrogenated castor oil but does not include a mild surfactant, such as Sodium Methyl Cocoyl Taurate and a glucoside, in an active amount; while C8 includes hydrogenated castor oil and Sodium Methyl Cocoyl Taurate, but includes Caprylyl/Capryl Glucoside rather than decyl glucoside (as in E14). While C7 does include Sodium Methyl Cocoyl Taurate as an ingredient, this amount is present in a residual amount and not in sufficient quantity to be an active ingredient. Further, C6, C7, or C8 do not include the Sodium Hydrolyzed Potato Starch Dodecenylsuccinate which is present in E14.

TABLE 9
Additional Examples

| E14 | C6 | C7 | C8 |
|---|---|---|---|
| Water | Water | Water | Water |
| Guar | Sodium | Sodium C14-16 | Cocamidopropyl |
| Hydroxypropyl- | Lauryl | Olefin | Betaine |
| trimonium | Ether | Sulfonate | |
| Chloride | Sulfate | | |
| | (15 | | |
| | wt %) | | |
| Sodium Hydrolyzed | | Cocamidopropyl | Sodium Methyl |
| Potato Starch | | Betaine | Cocoyl Taurate |
| Dodecenylsuccinate | | | |
| Coco-Glucoside | | Sodium Chloride | Coco-glucoside |
| (and) Hydrogenated | | | |
| Castor Oil | | | |
| Amodimethicone | | Curcuma Longa | Sodium Chloride |
| | | (Turmeric) Root | |
| | | Extract | |
| PEG-150 Distearate | | Hydrolyzed Collagen | Sodium Methyl 2- |
| | | (Hydrolysed) | sulfolaurate |
| Hexylene Glycol | | Cocamide Methyl | PEG-7 Glyceryl |
| | | MEA | Cocoate |
| Glycol Distearate | | Glycol Distearate | PEG-120 Methyl |
| | | | Glucose Dioleate |
| Sodium Methyl | | Dimethicone | Lanthanum |
| Cocoyl Taurate | | | Chloride |
| Cocamidopropyl | | Parfum | Glycine |
| Betaine | | | |
| Decyl Glucoside | | Benzyl Alcohol | Succinic Acid |

Silk

| | | |
|---|---|---|
| Sodium Benzoate | Acrylates Copolymer | Hydrolyzed Silk |
| | | (Hydrolysed) |
| Citric Acid | Guar Hydroxypropyl- | Sericin |
| | trimonium Chloride | |
| Sodium Hydroxide | Sodium Benzoate | Nymphaea Alba |
| | | Root Extract |
| Cocos Nucifera | Phenoxyethanol | Panthenol |
| (Coconut) Fruit | | |
| Extract | | |
| Hydrolyzed Milk | Glycerin | Caprylyl/Capryl |
| Protein | | Glucoside |
| Fragrance | Disodium EDTA | Citric Acid |
| | Polyquaternium-7 | Parfum |
| | Sodium Methyl | Sodium Benzoate |
| | Cocoyl Taurate | |
| | Hydrogenated Castor | Disodium 2- |
| | Oil (Hydrogenated) | sulfolaurate |
| | Butylene Glycol | Propylene Glycol |
| | Lauric Acid | Coconut Acid |
| | Citric Acid | Hydrogenated |
| | | Castor Oil |
| | | (Hydrogenated) |
| | Trideceth-10 | Glycol Distearate |
| | Trideceth-3 | Sodium Sulfate |
| | PPG-28-buteth-35 | Linalool |
| | Propylene Glycol | C12-18 |
| | | Fatty Acids |
| | | Methyl Esters |
| | | (Fatty) |
| | CI 19140 | Hexyl Cinnamal |
| | Ethylhexylglycerin | L-limonene |
| | Cocos Nucifera Oil | Citronellol |
| | (Coconut Oil | |
| | Derived) | |
| | Hydroxy- | Alpha-isomethyl |
| | acetophenone | Ionone |
| | CI 15985 | Sorbitol |
| | Malus Domestica | Butylene Glycol |
| | Fruit Extract | |
| | Hydrolyzed Milk | Polyquaternium-10 |
| | Protein (Hydrolysed) | |
| | 1,2-hexanediol | Sodium Hydroxide |
| | Tocopherol | Benzyl Alcohol |
| | | Sodium Acetate |
| | | Potassium Sorbate |
| | | Laureth-4 |
| | | Methylparaben |
| | | Propylparaben |

It should be understood that E14 is an augmentation of, and contains only slight modifications relative to, E1 as shown above. For example, E1 does not include the RTB agents, including but not limited to, Cocos Nucifera (Coconut) Fruit Extract, Hydrolyzed Milk Protein, and Fragrance, that are present in E14.

The treatment application process was as follows. For each application, a cleanser equal to ten % weight of the tress was applied. The cleanser was used on the wet tresses for 60 seconds, left on for 30 seconds, and then rinsed for 30 seconds under Intellifaucet water. The tresses were then blow-dried on medium heat for 5 minutes each to ensure drying. After the tresses were dried, they were scanned by ATR-FTIR spectroscopy to evaluate the lipid removal after ten, twenty, and thirty wash treatments. After each of the tenth, twentieth, and thirtieth was treatments, eighteen FTIR spectra were recorded.

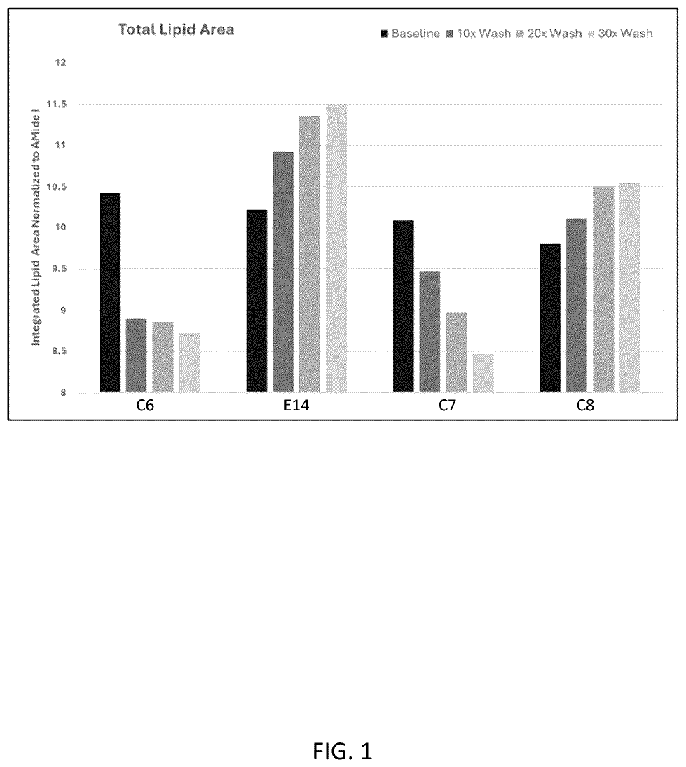

FIG. 1 illustrates values extracted from the ATR-FTIR spectra for the total lipid content of example formulations as described herein. FIG. 1 illustrates the decrease in lipid content from the baseline over each of 10×, 20×, and 30× washes for C6 and C7, and an increase in lipid content from the baseline over each of 10×, 20×, and 30× washes for E14 and C8. The results of FIG. 1 are illustrated in Table 9 below.

TABLE 10

| Treatment | Avg % Lipid Reduction |
|---|---|
| C6 | 15.25% |
| C7 | 11.12% |
| | Avg % Lipid Increase |
| E14 | 10.17% |
| C8 | 6.02% |

As shown in FIG. 1, the unexpected result of an observed stepwise increase in lipid content with each wash application of E14 results in a rise in lipid levels, indicating the persistence of lipids, i.e., the hydrogenated castor oil, on the hair surface attributed to a film deposition of E14 along the hair fibers. This creates a thin and resilient lipid layer, preventing damages related to multiple shampoo applications and further improving the lipid content along the hair fibers. Accordingly, this highlights the prolonged protective effect of E14, contributing to sustained resistance against de-lipidation even after multiple washing cycles. FIG. 1 further illustrates a similar effect, though to a much lesser degree, was observed with C8.

This phenomenon is not exhibited in C6. As shown in Table 9, C6 does not include any lipids that could be deposited on the hair fibers. Thus, a stepwise increase in lipid content would not be expected based on the application of C6.

This phenomenon is also not exhibited in C7 despite the presence of hydrogenated castor oil in the formulation of C7. This discrepancy in surfactants used between C7 and each of E14 and C8 appears to explain why C7 resulted in a decrease in lipid content over each wash application. As noted above, C7 includes Sodium C14-16 Olefin Sulfonate and Cocamidopropyl Betaine as surfactants, but does not include the Sodium Methyl Cocoyl Taurate in an active amount or a glucoside. In contrast, each of E14 and C8 include an active amount of Sodium Methyl Cocoyl Taurate as well as glucoside as surfactants, which appear to provide a synergistic effect of the stepwise increase in lipid content with each wash application.

While each of E14 and C8 present an observed stepwise increase in lipid content with each wash application, it should be noted that FIG. 1 illustrates that E14 presents this increase at a 1.6× greater rate than C8, as measured by the slopes of E1 and C8. C8 does not include a modified potato starch, such as the Sodium Hydrolyzed Potato Starch Dodecenylsuccinate, present in E14 as a conditioning agent, and further includes coco-glucoside rather than decyl glucoside, present in E14. The combination of the Sodium Hydrolyzed Potato Starch Dodecenylsuccinate and decyl glucoside appears to account for the additional advantage of a 1.6× increase in rate of lipid increase per wash application of E14 over C8.

In some examples, a system includes a hair cleansing formulation and a hair conditioning formulation. The hair cleansing formulation comprises a surfactant mixture comprising at least: an amphoteric surfactant, an anionic surfactant and a non-ionic surfactant; and water. The active materials present in the surfactant mixture amount for at least 10%, in weight %, of the hair cleansing formulation. The hair conditioning formulation comprises at least one conditioning agent; at least one emollient; and at least one non-ionic surfactant.

In another example, a method is provided. The method includes treating human hair with the system for hair care by applying the hair cleansing formulation to the human hair, rinsing the hair cleansing formulation from the human hair; and applying the hair conditioning formulation to the human hair.

In another example, a method is provided. The method includes reducing split ends in human hair by contacting a subject hair with the system for hair care.

In another example, a method is provided. The method includes reducing human hair breakage by contacting a subject hair with the system for hair care.

Further examples are described herein. Various examples further include one or more of the following:
wherein the hair cleansing formulation further comprises a superhydrophilic amphiphilic copolymer;
wherein the hair cleansing formulation further comprises a cationic polymer;
wherein, in the hair cleansing formulation, a ratio of the active materials between the amphoteric surfactant, the anionic surfactant, and the non-ionic surfactant is in the range (2-3):(0.5-1.5):(0.5-1.5);
wherein the amphoteric surfactant in the hair cleansing formulation is an amphocarboxylate selected among alkylamphoacetates; an alkylbetaines; an amidoalkyl betaines; an amidoalkyl sultaines; an amphophosphates; a phosphorylated imidazolines selected among phosphobetaines and pyrophosphobetaines; a carboxyalkyl alkyl polyamines; an alkylimino-dipropionates; an alkylamphoglycinates; an alkylamphoproprionates; a N-alkyl β-aminoproprionic acids; an alkylpolyamino carboxylates; and mixtures thereof;
wherein the anionic surfactant in the hair cleansing formulation is selected among: alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl laurates, fatty alkyl sulfoacetates, alkyl phosphates, and mixtures of two or more thereof;
wherein the non-ionic surfactant in the hair cleansing formulation is selected among: fatty alcohol acid or amide ethoxylates, monoglyceride ethoxylates, sorbitan ester ethoxylates, alkyl glucosides or polyglucosides, and mixtures thereof;
wherein the hair conditioning formulation further comprises at least one humectant;
wherein the hair conditioning formulation further comprises at least one emulsion stabilizer;
wherein, in the hair cleansing formulation: the surfactant mixture comprises: 20 to 25% of the amphoteric surfactant, wherein the amphoteric surfactant is cocamidopropylbetaine, 50 to 55% of the anionic surfactant, wherein the anionic surfactant is sodium methyl cocoyl taurate, and 23 to 28% of the non-ionic surfactant, wherein the non-ionic surfactant is decyl glucoside;
wherein the hair cleansing formulation further comprises: a starch-based polysaccharide derived from potato or tapioca modified with dodecenyl succinic anhydride; and a cationic polymer selected from Guar Hydroxypropyltrimonium Chloride, Starch Hydroxypropyltrimonium Chloride, Hydroxypropyl Guar Hydroxypropyltrimonium Chloride, or mixture thereof; and
wherein the hair conditioning formulation further comprises: 2.5 to 4% of the conditioning agent, wherein the conditioning agent is selected among behentrimonium chloride, stearamidopropyl dimethylamine, dimethicone, or mixtures thereof, 0.5 to 1% of the emollient, wherein the emollient is selected among Isopropyl myristate, Coco caprylate/caprate, or mixtures thereof, and to 0.5% of the non-ionic surfactant, wherein the non-ionic surfactant is selected among alkoxylated alcohols, ethoxylated alcohols, or combination thereof.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

1. A system for hair care comprising:
a hair cleansing formulation comprising:
a surfactant mixture comprising at least:
an amphoteric surfactant,
an anionic surfactant, and
a non-ionic surfactant; and
water, wherein active materials present in the surfactant mixture amount for at least 10%, in weight %, of the hair cleansing formulation; and
a hair conditioning formulation comprising:
a conditioning agent,
an emollient, and
a non-ionic surfactant.

2. The system for hair care of claim 1, wherein the hair cleansing formulation further comprises a superhydrophilic amphiphilic copolymer.

3. The system for hair care of claim 1, wherein the hair cleansing formulation further comprises a cationic polymer.

4. The system for hair care of claim 1, wherein, in the hair cleansing formulation, a ratio of the active materials between the amphoteric surfactant, the anionic surfactant, and the non-ionic surfactant is in a range of (2-3):(0.5-1.5):(0.5-1.5).

5. The system for hair care of claim 1, wherein the amphoteric surfactant in the hair cleansing formulation is an amphocarboxylate selected among alkylamphoacetates; an alkylbetaines; an amidoalkyl betaines; an amidoalkyl sultaines; an amphophosphates; a phosphorylated imidazolines selected among phosphobetaines and pyrophosphobetaines; a carboxyalkyl alkyl polyamines; an alkylimino-dipropionates; an alkylamphoglycinates; an alkylamphoproprionates; a N-alkyl β-aminoproprionic acids; an alkylpolyamino carboxylates; and mixtures thereof.

6. The system for hair care of claim 1, wherein the anionic surfactant in the hair cleansing formulation is selected among: alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl laurates, fatty alkyl sulfoacetates, alkyl phosphates, and mixtures of two or more thereof.

7. The system for hair care of claim 1, wherein the non-ionic surfactant in the hair cleansing formulation is selected among: fatty alcohol acid or amide ethoxylates, monoglyceride ethoxylates, sorbitan ester ethoxylates, alkyl glucosides, polyglucosides, and mixtures thereof.

8. The system for hair care of claim 1, wherein the hair conditioning formulation further comprises at least one humectant.

9. The system for hair care of claim 1, wherein the hair conditioning formulation further comprises at least one emulsion stabilizer.

10. The system for hair care of claim 1, wherein, in the hair cleansing formulation:
the surfactant mixture comprises:
20 to 25% of the amphoteric surfactant, wherein the amphoteric surfactant is cocamidopropylbetaine,
50 to 55% of the anionic surfactant, wherein the anionic surfactant is sodium methyl cocoyl taurate, and
23 to 28% of the non-ionic surfactant, wherein the non-ionic surfactant is decyl glucoside.

11. The system for hair care of claim 10, wherein the hair cleansing formulation further comprises:
a starch-based polysaccharide derived from potato or tapioca modified with dodecenyl succinic anhydride; and
a cationic polymer selected from Guar Hydroxypropyltrimonium Chloride, Starch Hydroxypropyltrimonium Chloride, Hydroxypropyl Guar Hydroxypropyltrimonium Chloride, or mixture thereof.

12. The system for hair care of claim 1, wherein the hair conditioning formulation further comprises:
2.5 to 4% of the conditioning agent, wherein the conditioning agent is selected among behentrimonium chloride, stearamidopropyl dimethylamine, dimethicone, or mixtures thereof,
0.5 to 1% of the emollient, wherein the emollient is selected among Isopropyl myristate, Coco caprylate/caprate, or mixtures thereof, and
to 0.5% of the non-ionic surfactant, wherein the non-ionic surfactant is selected among alkoxylated alcohols, ethoxylated alcohols, or combination thereof.

13. A method of treating human hair with the system for hair care of claim 1, the method comprising:
applying the hair cleansing formulation to the human hair,
rinsing the hair cleansing formulation from the human hair;
applying the hair conditioning formulation to the human hair; and
rinsing the hair conditioning formulation from the human hair.

14. A method of reducing split ends in human hair, the method comprising contacting a subject hair with the system for hair care of claim 1.

15. A method of reducing human hair breakage, the method comprising contacting a subject hair with the system for hair care of claim 1.
